(19) United States
(12) Patent Application Publication
(10) Pub. No.: US 2011/0028780 A1
(43) Pub. Date: Feb. 3, 2011

(54) Methods Of Producing Pluripotent Stem Cell-Generated Embryos, And Animals Derived Therefrom

(76) Inventor: Robert Lanza, Clinton, MA (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(21) Appl. No.: 12/809,704

(22) PCT Filed: Dec. 19, 2008
(86) PCT No.: PCT/US08/13906

§ 371 Date: Oct. 15, 2010

Related U.S. Application Data
(60) Provisional application No. 61/066,743, filed on Feb. 22, 2008.
(60) Provisional application No. 61/008,384, filed on Dec. 19, 2007.

(51) Int. Cl. A61B 17/425

(52) U.S. Cl. 600/33

(57) ABSTRACT

Methods for generating embryos using pluripotent stem cells are provided. The subject methods include methods for generating chimeric embryos, wherein only a subset of the cells of each embryo are genetically identical to the pluripotent stem cells used in the generation process. The subject methods also include methods for generating embryos that are identical or are essentially genetic clones of the pluripotent stem cells (e.g., the resulting embryos are substantially identical, genetically, to the pluripotent stem cells used in the generation process).

### RELATED APPLICATIONS

This application claims the benefit of priority from U.S. provisional application Nos. 61/008,384, filed Dec. 19, 2007, and 61/066,743, filed Feb. 22, 2008. The specifications of each of the foregoing applications are hereby incorporated by reference in their entirety.

### BACKGROUND

Embryonic stem cells and other pluripotent stem cells have numerous uses in the study of stem cell biology and in developing therapeutics. The application of embryonic stem cell technology has been hindered by legal restrictions limiting federal funding for human embryonic stem cell research, as well as technical obstacles in obtaining and testing embryonic stem cells from some non-laboratory animals.

However, the recent development of methods for generating pluripotent stem cells by reprogramming somatic cells provides additional sources of pluripotent stem cells with embryonic stem cell-like properties. Such cells have a variety of in vitro and in vivo uses.

### SUMMARY OF THE INVENTION

There is justifiable excitement surrounding the successful induction of pluripotent stem cells from human and non-human fibroblasts. The ability to produce pluripotent stem cells that share many of the fundamental properties of embryonic stem cells can alleviate the dependence of the stem cell community on oocytes, certain embryonic material, and the ability to generate embryonic stem cell lines from blastocysts or via somatic cell nuclear transfer.

This technology has a variety of applications to the study of stem cell biology, the development of therapeutics, and the identification of factors that can be used to influence stem cell behavior. Additionally, this technology can be used, as described herein, to produce embryos (pluripotent stem cell-generated embryos). Such embryos can be used in the area of conservation biology to help produce embryos and animals of a desired genetic make-up, to help maintain endangered species, to help maintain the genetic diversity of geographically isolated species, or even to reintroduce a previously extinct species. Further, such embryos have numerous applications in the agricultural context. For example, such embryos can be used to produce livestock of a desired genetic make-up.

Such embryos can also be used to study early development and the interactions between the embryo, placenta, and uterine environment, including the level of species specificity or relatedness of the in utero environment required for early development and gestation.

The invention provides methods for combining pluripotent stem cells with donor diploid or tetraploid embryos.

In one aspect, the invention provides a method of producing a pluripotent stem cell-generated embryo. The method comprises providing at least one pluripotent stem cell, and combining the at least one pluripotent stem cell with a diploid or tetraploid donor embryo to generate a diploid or tetraploid donor embryo comprising at least one pluripotent stem cell. The diploid or tetraploid donor embryo comprising said at least one pluripotent stem cell is then cultured and transferred to a surrogate female animal. The surrogate female animal gestates the embryo to produce a pluripotent stem cell-generated embryo.

In certain embodiments, the at least one pluripotent stem cell is at least one, at least two, at least three, at least four, at least five, at least six, at least seven, at least eight, at least nine, or at least ten pluripotent stem cells. In other embodiments, the at least one pluripotent stem cell is greater than ten, greater than fifteen, greater than twenty, or greater than fifty pluripotent stem cells. In other embodiments, the at least one pluripotent stem cell is greater than 100 pluripotent stem cells.

In certain embodiments, the pluripotent stem cell is an embryonic stem cell or embryo-derived cell. In other embodiments, the pluripotent stem cell is an induced pluripotent stem cell. In certain embodiments, the pluripotent stem cell is an induced pluripotent stem cell produced by expressing or activating/inducing the expression of one or more reprogramming factors in a somatic cell. In certain embodiments, the somatic cell is a fibroblast, such as a dermal fibroblast, synovial fibroblast, or lung fibroblast. In other embodiments, the somatic cell is not a fibroblast, but rather is a non-fibroblastic somatic cell. In certain embodiments, the somatic cell is reprogrammed by expressing or activating/inducing the expression of at least two reprogramming factors, at least three reprogramming factors, or four reprogramming factors. In other embodiments, the somatic cell is reprogrammed by expressing or activating/inducing the expression of at least four, at least five, or at least six reprogramming factors. In certain embodiments, the reprogramming factors are selected from Oct 3/4, Sox2, Nanog, Lin28, c-Myc, and Klf4. In other embodiments, the set of reprogramming factors expressed or activated includes at least one, at least two, at least three, or at least four of the foregoing list of reprogramming factors, and optionally includes one or more other reprogramming factors.

In certain embodiments, reprogramming factors are expressed in the somatic cell by infection using a viral vector, such as a retroviral vector or a lentiviral vector. In other embodiments, reprogramming factors are expressed in the somatic cell using a non-integrative vector, such as an episomal plasmid or a non-integrative viral vector. When reprogramming factors are expressed using non-integrative vectors, the factors can be expressed in the cells using, for example, infection, electroporation, transfection, or transformation of the somatic cells with the vectors.

In certain embodiments, expression of one or more reprogramming factors is activated/induced by, for example, the use of small organic molecules, cytoplasm, or other agents.

In certain embodiments, the pluripotent stem cells are generated from somatic cells, and the somatic cells are selected from embryonic, fetal, neonatal, juvenile, or adult cells.

In certain embodiments, combining the pluripotent stem cells and the donor embryo comprises injecting the stem cells into the donor embryo. In other embodiments, combining the pluripotent stem cells and the donor embryo comprises aggregating the pluripotent stem cells with the donor embryo.

In certain embodiments, the donor embryo is a diploid donor embryo, and the pluripotent stem cell-generated embryo is a chimeric embryo comprising cells contributed from the pluripotent stem cells and cells contributed from the donor embryo. In certain embodiments, the chimeric embryo comprises cells genetically identical to the pluripotent stem cell and cells genetically identical to the donor embryo. In certain embodiments, the cells from or genetically identical to the pluripotent stem cells contribute to the germ line of the pluripotent stem cell-generated embryo. In certain embodiments, “genetically identical” is assessed with respect to the original genotype of the cell, and without reference to the expression or integration of reprogramming factors.

In other embodiments, the donor embryo is a tetraploid donor embryo, and substantially all of the cells of the pluripotent stem cell-generated embryo are from the pluripotent stem cells. In certain embodiments, the donor embryo is a tetraploid donor embryo, and substantially all of the cells of the pluripotent stem cell-generated embryo are genetically identical to the pluripotent stem cell. In certain embodiments, “genetically identical” is assessed with respect to the original genotype of the cell, and without reference to the expression or integration of reprogramming factors. The tetraploid donor embryo does not substantially contribute to the developing embryo itself, it contributes to the placenta and extra-embryonic membranes.

The pluripotent stem cell can be derived from any species. For example, in certain embodiments, the pluripotent stem cell is derived from (produced using cells from) a human, a non-human primate, a rat, a mouse, a hamster, a gerbil, a hamster, a rabbit, a dog (wild or domestic), a cat (wild or domestic), a pig, a cow, a horse, a zebra, a goat, a bear, a squirrel, an elephant, a panda, a marine mammal, and the like. In certain embodiments, the species is an endangered species. In certain embodiments, the species is a currently extinct species. In certain embodiments, the species is an animal used in an agricultural or commercial setting (e.g., livestock). By way of non-limiting example, livestock includes goats, sheep, horses (farm or racing quality), pigs, cattle, bison, elk, alpaca, llama, emu, and the like. In certain embodiments, the species is an animal typically maintained in a zoo. When the pluripotent stem cell is an induced pluripotent stem cell, the species refers to the species from which the somatic cell was obtained. In certain embodiments, the species is not a human.

The diploid or tetraploid embryos can be generated from any species. For example, in certain embodiments, the diploid or tetraploid embryos are generated from (produced using cells from) a human, a non-human primate, a rat, a mouse, a hamster, a gerbil, a hamster, a rabbit, a dog (wild or domestic), a cat (wild or domestic), a pig, a cow, a horse, a zebra, a goat, a bear, a squirrel, an elephant, a panda, a marine mammal, and the like. In certain embodiments, the species is an endangered species. In certain embodiments, the species is an animal used in an agricultural or commercial setting (e.g., livestock). By way of non-limiting example, livestock includes goats, sheep, horses (farm or racing quality), pigs, cattle, bison, elk, alpaca, llama, emu, and the like. In certain embodiments, the species is an animal typically maintained in a zoo. The embryo can be produced in vitro by combining or otherwise fusing or activating ova and sperm from animals of the species of interest. Alternatively, the embryo can be flushed or otherwise removed from the fallopian tube or uterus of a pregnant animal. In certain embodiments, the species is not a human.

In certain embodiments, the embryo is a tetraploid embryo and tetraploidy is induced in vitro using, for example, chemical, physical, or electrical stimulation.

In certain embodiments, the at least one pluripotent stem cell and the diploid or tetraploid donor embryo are from the same species. In other embodiments, the at least one pluripotent stem cell and the diploid or tetraploid donor embryo are from differing species. In certain embodiments, the pluripotent stem cell and the diploid or tetraploid donor embryo are from differing species, but the differing species are related in some way. By way of example, the species are from the same genus, family, or order. By way of another example, the species are of a similar size (either as adults or as embryos).

In certain embodiments, the surrogate female is of the same species as the diploid or tetraploid donor embryo. In certain embodiments, the pluripotent stem cells, the surrogate mother, and the diploid or tetraploid donor embryo are all of the same species. In certain embodiments, the surrogate female is genetically related to the diploid or tetraploid donor embryo. In certain embodiments, the surrogate female is the same animal from whose ova the diploid or tetraploid donor embryo was generated. In certain embodiments, the surrogate female and the diploid or tetraploid donor embryo are from differing species, but the differing species are related in some way. By way of example, the species are from the same genus, family, or order. By way of another example, the species are of a similar size (either as adults or as embryos).

In certain embodiments, the surrogate female gestates the embryo for some period other than full term, and the embryo (the pluripotent stem cell-generated embryo) is harvested from the female for further study or analysis. In certain embodiments, the surrogate female animal gestates said embryo (the pluripotent stem cell-generated embryo) to term and delivers a pluripotent stem cell-generated animal.

In certain embodiments, the pluripotent stem cell is from an endangered species, and the diploid or tetraploid donor embryo is from a different species. In certain embodiments, the pluripotent stem cell is from an endangered species, and the diploid or tetraploid donor embryo is from a different but related species.

In certain embodiments, the pluripotent stem cell is genetically manipulated prior to combining said pluripotent stem cell with said donor embryo. In certain embodiments, genetically manipulated does not refer to the expression of reprogramming factors necessary to generate a pluripotent stem cell from a somatic cell. Rather, genetically manipulated refers to other manipulations of the genetic material of the cell, other than manipulations necessary to generate the pluripotent stem cell. In certain embodiments, genetically manipulating comprises correcting a genetic defect in the cell. In other embodiments, genetically manipulating comprises reducing immunocomplexity to reduce the chance of provoking an immune response in the host embryo or mother. In other embodiments, genetically manipulating comprises expression of a positive and/or negative selection marker and/or detectable label to allow identification and/or visualization of the pluripotent stem cell.

In a related aspect, the pluripotent stem cell is combined with a diploid donor embryo to generate a diploid donor embryo comprising at least one pluripotent stem cell. As noted above, in such embodiments, the generated embryo or animal is chimeric, wherein a portion of the cells of the embryo or animal are genetically identical to the pluripotent stem cell and a portion of the cells of the embryo or animal are genetically identical to the donor diploid embryo. In certain embodiments, chimeric embryos or animals can be used or can be bred to produce embryos in which an increasing percentage (or even substantially all) of the embryo is genetically related to or a clone of the pluripotent stem cell. Alternatively, chimeric embryos or animals in which the pluripotent stem cell contributed to the germline (e.g., sperm and/or ova are genetically identical to the pluripotent stem cell) can be selected. Rather than breeding these chimeric animals, the sperm and/or ova can be obtained, from either the chimeric embryo or chimeric animal, and used to produce a fertilized embryo in vitro. Note that the sperm and ova can be obtained from the same animal or from differing animals. Given that the sperm and ova obtained from the chimeric embryo or animal are genetically identical to the pluripotent stem cell, the embryo produced following in vitro fertilization using these gametes will be genetically identical (a clone) of the pluripotent stem cell. This clonal embryo, produced using gametes from the chimeric embryo, can be transferred to a surrogate mother to gestate the embryo, as described throughout the specification.

In certain embodiments of this related aspect of the invention, the pluripotent stem cell is modified with a detectable marker to facilitate identification of gametes in the chimeric embryo that are genetically identical to the pluripotent stem cell. In this way, regardless of the level of efficiency with which pluripotent stem cells contribute to the germ line of a chimeric embryo or animal, a skilled practitioner can readily identify the appropriate sperm and/or ova, and use such cells in methods of in vitro fertilization.

In another aspect, the invention provides a method of generating pluripotent stem cells from an endangered species. The method comprises taking a tissue sample, such as a dermal fibroblast sample, from an animal Of an endangered species. Cells from the sample are used to make pluripotent stem cells, for example, by expressing reprogramming factors (making iPS cells). The pluripotent stem cells are then combined with a diploid or tetraploid embryo, as described above. In certain embodiments, the pluripotent stem cells are combined with an embryo of the same endangered species. When used in this manner, the method helps overcome problems associated with species that don't breed frequently or easily. Alternatively, the pluripotent stem cells can be combined with an embryo of another species. Preferably, the other species is selected based on a level of evolutionary or structural similarities to help ensure successful embryonic development (e.g., the other species is of the same genus, family, or order). As described above, the resulting embryo can be transferred to a surrogate female to gestate the developing embryo.

In another aspect, the invention provides a method for identifying the level of cross-species flexibility of reprogramming factors. In other words, the invention provides a method for assessing the level of species difference tolerated by reprogramming factors before they fail to function to reprogram a somatic cell to a pluripotent cell. The method comprises evaluating reprogramming factors in cells from increasingly divergent or otherwise unrelated organisms, and evaluating at what point the reprogramming factors no longer induce reprogramming. If reprogramming factors from humans or mice fail to work in cells of a particular species, related reprogramming factors can be identified and cloned from other species that are more evolutionarily related to the organism in which reprogramming is desired. For example, cDNA libraries can be readily made from, for example, discarded testicular or other tissue of another species (e.g., bovine, ovine, pig, goat, horse, etc.). Reprogramming factors can be readily cloned using standard molecular biological approaches and used to induce reprogramming in cells of additional species, in the event that cDNA encoding human or mice factors fails to function across a certain evolutionary distance.

In a related aspect, the invention provides methods for generating iPS cells from non-mouse and non-human species. In certain embodiments, the invention provides methods for generating iPS cells from an endangered species.

In certain embodiments, the iPS cells are used in screening assays to identify factors that can be used to influence the proliferation, differentiation, or survival of iPS cells. In other embodiments, the iPS cells are used to produce differentiated cell types, in vitro or in vivo. In other embodiments, the iPS cells are used to produce an embryo by combining the cells with a diploid or tetraploid embryo.

In another aspect, the foregoing methods can be applied with human cells and embryos. When used in this manner, the invention provides a method to treat a genetic defect in an embryo. Additionally or alternatively, the invention provides additional options for assisted reproduction. For example, a couple whose children need to be conceived using either donor sperm or donor egg would have the ability to combine some of their own genetic material (or the genetic material from whichever partner would not otherwise be represented in the developing embryo) with that of the embryo produced using donor egg and/or sperm. In this way, couples who can not presently have the genetics of both partners represented in their children, would have a mechanism for doing so.

In another aspect, the foregoing methods can be applied to generate animals that are genetically related or substantially identical to existing or deceased animals. For example, the method can be applied to the production of livestock and can be used to help propagate desirable traits. By way of another example, the method can be applied to the production of horses and can be used to help propagate the genetics of thoroughbred race horses. By way of another example, the method can be used with domestic animals to help pet owners have animals that share the genetics of an ailing, deceased, or still living pet.

The invention contemplates all suitable combinations of any of the forgoing or following aspects and embodiments of the invention.

### DETAILED DESCRIPTION OF THE INVENTION

In order that the invention herein described may be fully understood, the following detailed description is set forth. Various embodiments of the invention are described in detail and may be further illustrated by the provided examples.

All publications, patents, patent publications and applications and other documents mentioned herein are incorporated by reference in their entirety.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

Throughout this specification, the word “comprise” or variations such as “comprises” or “comprising” will be understood to imply the inclusion of a stated integer or groups of integers but not the exclusion of any other integer or group of integers.

The term “embryonic stem cells” refers to embryo-derived cells. More specifically it refers to cells isolated from the inner cell mass of blastocysts or morulae, including those that have been serially passaged as cell lines. The term also includes cells isolated from one or more blastomeres of an embryo, preferably without destroying the remainder of the embryo. The term also includes cells produced by somatic cell nuclear transfer, even when non-embryonic cells are used in the process.

The term “embryonic stem cells” (ES cells) refers to embryo-derived cells and is used herein as it is used in the art. This term includes cells derived from the inner cell mass of human blastocysts or morulae, including those that have been serially passaged as cell lines. When used to refer to cells from humans, the term human embryonic stem cell (hES) cell is used. The ES cells may be derived from fertilization of an egg cell with sperm, as well as using DNA, nuclear transfer, parthenogenesis, or by means to generate ES cells with homozygosity in the HLA region. ES cells are also cells derived from a zygote, blastomeres, or blastocyst-staged mammalian embryo produced by the fusion of a sperm and egg cell, nuclear transfer, parthenogenesis, androgenesis, or the reprogramming of chromatin and subsequent incorporation of the reprogrammed chromatin into a plasma membrane to produce a cell. Embryonic stem cells, regardless of their source or the particular method use to produce them, can be identified based on (i) the ability to differentiate into cells of all three germ layers, (ii) expression of at least Oct-4 and alkaline phosphatase, and (iii) ability to produce teratomas when transplanted into immunodeficient animals.

As used herein, the term “pluripotent stem cells” includes embryonic stem cells, embryo-derived stem cells, and induced pluripotent stem cells, regardless of the method by which the pluripotent stem cells are derived. Pluripotent stem cells are defined functionally as stem cells that are: (a) capable of inducing teratomas when transplanted in immunodeficient (SCID) mice; (b) capable of differentiating to cell types of all three germ layers (e.g., can differentiate to ectodermal, mesodermal, and endodermal cell types); and (c) express one or more markers of embryonic stem cells (e.g., express Oct 4, alkaline phosphatase, SSEA-3 surface antigen, SSEA-4 surface antigen, nanog, TRA-1-60, TRA-1-81, SOX2, REX1, etc). Exemplary pluripotent stem cells can be generated using, for example, methods known in the art. Exemplary pluripotent stem cells include embryonic stem cells derived from the ICM of blastocyst stage embryos, as well as embryonic stem cells derived from one or more blastomeres of a cleavage stage or morula stage embryo (optionally without destroying the remainder of the embryo). Such embryonic stem cells can be generated from embryonic material produced by fertilization or by asexual means, including somatic cell nuclear transfer (SCNT), parthenogenesis, and androgenesis. Further exemplary pluripotent stem cells include induced pluripotent stem cells (iPS cells) generated by reprogramming a somatic cell by expressing or activating/inducing the expression of a combination of factors (herein referred to as reprogramming factors). iPS cells can be generated using fetal, postnatal, newborn, juvenile, or adult somatic cells. In certain embodiments, factors that can be used to reprogram somatic cells to pluripotent stem cells include, for example, a combination of Oct4 (sometimes referred to as Oct 3/4), Sox2, c-Myc, and Klf4. In other embodiments, factors that can be used to reprogram somatic cells to pluripotent stem cells include, for example, a combination of Oct 4, Sox2, Nanog, and Lin28. In other embodiments, somatic cells are reprogrammed by expressing at least two reprogramming factors, at least three reprogramming factors, or four reprogramming factors. In other embodiments, additional reprogramming factors are identified and used alone or in combination with one or more known reprogramming factors to reprogram a somatic cell to a pluripotent stem cell.

The pluripotent stem cells can be from any species. Embryonic stem cells have been successfully derived in, for example, mice, multiple species of non-human primates, and humans, and embryonic stem-like cells have been generated from numerous additional species. Thus, one of skill in the art can generate embryonic stem cells and embryo-derived stem cells from any species, including but not limited to, human, non-human primates, rodents (mice, rats), ungulates (cows, sheep, etc), dogs (domestic and wild dogs), cats (domestic and wild cats such as lions, tigers, cheetahs), rabbits, hamsters, gerbils, squirrel, guinea pig, goats, elephants, panda (including giant panda), pigs, raccoon, horse, zebra, marine mammals (dolphin, whales, etc.) and the like. In certain embodiments, the species is an endangered species. In certain embodiments, the species is a currently extinct species. In certain embodiments, the species is an animal frequently used in an agricultural or commercial setting (e.g., livestock). By way of non-limiting example, livestock includes goats, sheep, horses (farm or racing quality), pigs, cattle, bison, elk, alpaca, llama, emu, and the like. In certain embodiments, the species is not a human.

Similarly, iPS cells can be from any species. iPS cells have been successfully generated using mouse and human cells. iPS cells have been successfully generated using embryonic, fetal, newborn, and adult tissue. Accordingly, one can readily generate iPS cells using a donor cell from any species and/or stage of development. Thus, one can generate iPS cells from any species, including but not limited to, human, non-human primates, rodents (mice, rats), ungulates (cows, sheep, etc), dogs (domestic and wild dogs), cats (domestic and wild cats such as lions, tigers, cheetahs), rabbits, hamsters, goats, elephants, panda (including giant panda), pigs, raccoon, horse, zebra, marine mammals (dolphin, whales, etc.) and the like. In certain embodiments, the species is an endangered species. In certain embodiments, the species is a currently extinct species. In certain embodiments, the species is an animal frequently used in an agricultural or commercial setting (e.g., livestock). By way of non-limiting example, livestock includes goats, sheep, horses (farm or racing quality), pigs, cattle, bison, elk, alpaca, llama, emu, and the like.

Induced pluripotent stem cells can be generated using, as a starting point, virtually any somatic cell of any developmental stage. For example, the cell can be from an embryo, fetus, neonate, juvenile, or adult donor. Exemplary somatic cells that can be used include fibroblasts, such as dermal fibroblasts obtained by a skin sample or biopsy, synoviocytes from synovial tissue, foreskin cells, cheek cells, or lung fibroblasts. Although skin and cheek provide a readily available and easily attainable source of appropriate cells, virtually any cell can be used. In certain embodiments, the somatic cell is not a fibroblast.

The invention provides methods for combining pluripotent stem cells with diploid or tetraploid embryos to produce pluripotent stem cell-generated embryos. By combining, is meant that the pluripotent stem cells and the diploid or tetraploid embryos are physically combined, such as by injection, microsurgical implantation, aggregation, electroporation, and the like. Once combined, the diploid or tetraploid embryo comprising pluripotent stem cells is cultured and, subsequently, the embryo is transferred to a surrogate female. Specifically, the embryo is transferred to a pseudopregnant female where the embryo can implant into the uterus and continue to develop. The embryo can be delivered at or near term via cesarean section (with or without sacrificing the surrogate female) or via vaginal delivery. In other embodiments, the embryo is harvested from the uterus before reaching term, for example before viability, for further study or to carry out screening assays.

Note that the pluripotent stem cells can be, for example, ES cells or induced pluripotent stem cells. Induced pluripotent stem cells can be produced by expressing or activating/inducing the expression of a combination of reprogramming factors in a somatic cell. In certain embodiments, at least one reprogramming factor is expressed in a somatic cell to successfully reprogram the somatic cell. In certain embodiments, at least two reprogramming factors are expressed in a somatic cell to successfully reprogram the somatic cell. In other embodiments, at least three reprogramming factors are expressed in a somatic cell to successfully reprogram the somatic cell. In other embodiments, at least four reprogramming factors are expressed in a somatic cell to successfully reprogram the somatic cell.

The present invention has a variety of uses. Pluripotent stem cells can be used to study basic developmental and stem cell biology, and can be used in screening assays to identify factors that influence the proliferation, differentiation, or survival of pluripotent stem cells. Pluripotent stem cells can also be used to generate differentiated cell types, in vitro or in vivo. By providing additional methods for producing pluripotent stem cells, including iPS cells using non-mouse and non-human cells, the invention provides additional sources of pluripotent stem cells that can be used in vitro and in vivo.

Additionally, pluripotent stem cells can be used to generate embryos (pluripotent stem cell-generated embryos). Such embryos can be used to produce chimeric or cloned animals. Alternatively, the embryos can be used in the study of basic developmental biology, for example, in the study of the compatibilities between the placental and uterine environments of different species of animals. The embryos can also be used as a source of tissue of a particular lineage or as a source of materials for screening assays.

When the methods of the present invention are used to produce embryos, the methods can help circumvent the need for producing embryonic stem cells as a mechanism to produce chimeric or clonal embryos The methods of the present invention can also be used to study and help maintain fragile or otherwise endangered animals, and can also be used to help re-introduce previously extinct species of animals. The methods of the present invention can also be used in the agricultural setting to help produce livestock having a desired genetic make-up. By way of example, these methods can be used to produce genetically similar or clonal embryos and animals, such as goats, sheep, horses (farm or racing quality), pigs, cattle, bison, elk, alpaca, llama, emu, and the like. The methods of the present invention can be used in the zoo setting to help produce or maintain animal species, for example, animals that do not breed well in captivity.

(i) Detailed Description of the Methods for Producing Pluripotent Stem Cell-Generated Embryos

In one aspect, the invention provides a method of producing a pluripotent stem cell-generated embryo. The method comprises providing at least one pluripotent stem cell, and combining the at least one pluripotent stem cell with a diploid or tetraploid donor embryo to generate a diploid or tetraploid donor embryo comprising at least one pluripotent stem cell. The diploid or tetraploid donor embryo comprising said at least one pluripotent stem cell is then cultured and transferred to a surrogate female animal. The surrogate female animal gestates the embryo to produce a pluripotent stem cell-generated embryo.

In certain embodiments, the at least one pluripotent stem cell is at least one, at least two, at least three, at least four, at least five, at least six, at least seven, at least eight, at least nine, or at least ten pluripotent stem cells. In other embodiments, the at least one pluripotent stem cell is greater than ten, greater than fifteen, greater than twenty, or greater than fifty pluripotent stem cells. In other embodiments, the at least one pluripotent stem cell is greater than 100 pluripotent stem cells.

In certain embodiments, the pluripotent stem cell is an embryonic stem cell or embryo-derived cell. In other embodiments, the pluripotent stem cell is an induced pluripotent stem cell. In certain embodiments, the pluripotent stem cell is an induced pluripotent stem cell produced by expressing or activating/inducing the expression of one or more reprogramming factors in a somatic cell. In certain embodiments, the somatic cell is a fibroblast, such as a dermal fibroblast, synovial fibroblast, or lung fibroblast. In other embodiments, the somatic cell is not a fibroblast, but rather is a non-fibroblastic somatic cell. In certain embodiments, the somatic cell is reprogrammed by expressing at least two reprogramming factors, at least three reprogramming factors, or four reprogramming factors. In other embodiments, the somatic cell is reprogrammed by expressing at least four, at least five, or at least six reprogramming factors. In certain embodiments, the reprogramming factors are selected from Oct 3/4, Sox2, Nanog, Lin28, c-Myc, and Klf4. In other embodiments, the set of reprogramming factors expressed includes at least one, at least two, at least three, or at least four of the foregoing list of reprogramming factors, and optionally includes one or more other reprogramming factors.

In certain embodiments, reprogramming factors are expressed in the somatic cell by infection using a viral vector, such as a retroviral vector or a lentiviral vector. In other embodiments, reprogramming factors are expressed in the somatic cell using a non-integrative vector, such as an episomal plasmid or a non-integrative viral vector. When reprogramming factors are expressed using non-integrative vectors, the factors can be expressed in the cells using infection, electroporation, transfection, or transformation of the somatic cells with the vectors.

In certain embodiments, expression of one or more reprogramming factors is activated/induced by, for example, the use of small organic molecules, cytoplasm, or other agents.

In certain embodiments, the pluripotent stem cells are generated from somatic cells, and the somatic cells are selected from embryonic, fetal, neonatal, juvenile, or adult cells.

In certain embodiments, combining the pluripotent stem cells and the donor embryo comprises injecting the stem cells into the donor embryo. In other embodiments, combining the pluripotent stem cells and the donor embryo comprises aggregating the pluripotent stem cells with the donor embryo.

In certain embodiments, the donor embryo is a diploid donor embryo, and the pluripotent stem cell-generated embryo is a chimeric embryo comprising cells contributed from the pluripotent stem cells and cells contributed from the donor embryo. In certain embodiments, the chimeric embryo comprises cells genetically identical to the pluripotent stem cell and cells genetically identical to the donor embryo. In certain embodiments, the cells from or genetically identical to the pluripotent stem cells contribute to the germ line of the pluripotent stem cell-generated embryo. In certain embodiments, such germ line cells (germ line cells genetically identical to the pluripotent stem cells) are specifically selected from an embryo or adult chimeric animal, and combined in vitro using standard in vitro fertilization techniques to produce a clonal embryo (a pluripotent stem-cell generated embryo genetically identical to the pluripotent stem cells). Such clonal pluripotent stem cell generated embryos can be transferred to a surrogate mother for gestation. In certain embodiments, “genetically identical” is assessed with respect to the original genotype of the cell, and without reference to the expression or integration of reprogramming factors.

In other embodiments, the donor embryo is a tetraploid donor embryo, and substantially all of the cells of the pluripotent stem cell-generated embryo are from the pluripotent stem cells. In such embodiments, the tetraploid donor embryo contributes to the placenta and the extra-embryonic tissue, rather than to the developing embryo itself. In certain embodiments, the donor embryo is a tetraploid donor embryo, and substantially all of the cells of the pluripotent stem cell-generated embryo are genetically identical to the pluripotent stem cell. In certain embodiments, “genetically identical” is assessed with respect to the original genotype of the cell, and without reference to the expression or integration of reprogramming factors.

The pluripotent stem cell can be derived from any species. For example, in certain embodiments, the pluripotent stem cell is derived from (produced using cells from) a human, a non-human primate, a rat, a mouse, a hamster, a gerbil, a hamster, a rabbit, a dog (wild or domestic), a cat (wild or domestic), a pig, a cow, a horse, a zebra, a goat, a bear, a squirrel, an elephant, a lion, a tiger, a giraffe, a panda, a marine mammal, and the like. In certain embodiments, the species is an endangered species. In certain embodiments, the species is a currently extinct species. In certain embodiments, the species is an animal frequently used in an agricultural or commercial setting (e.g., livestock). By way of non-limiting example, livestock includes goats, sheep, horses (farm or racing quality), pigs, cattle, bison, elk, alpaca, llama, emu, and the like. In certain embodiments, the species is an animal typically maintained in a zoo. When the pluripotent stem cell is an induced pluripotent stem cell, the species from which the cell is derived refers to the species from which the somatic cell was obtained.

The diploid or tetraploid embryos can be generated from any species. For example, in certain embodiments, the diploid or tetraploid embryos are generated from (produced using cells from) a human, a non-human primate, a rat, a mouse, a hamster, a rabbit, a gerbil, a dog (wild or domestic), a cat (wild or domestic), a pig, a cow, a horse, a zebra, a goat, a bear, a squirrel, an elephant, a lion, a tiger, a giraffe, a panda, a marine mammal, and the like. In certain embodiments, the species is an endangered species. In certain embodiments, the species is an animal frequently used in an agricultural or commercial setting (e.g., livestock). By way of non-limiting example, livestock includes goats, sheep, horses (farm or racing quality), pigs, cattle, elk, bison, alpaca, llama, emu, and the like. In certain embodiments, the species is an animal typically maintained in a zoo. The embryo can be produced in vitro by combining ova and sperm from animals of the species of interest. Alternatively, the embryo can be flushed or otherwise removed from the fallopian tube or uterus of a pregnant animal.

In certain embodiments, the embryo is a tetraploid embryo and tetraploidy is induced in vitro using, for example, chemical, physical, or electrical stimulation.

In certain embodiments, the at least one pluripotent stem cell and the diploid or tetraploid donor embryo are from the same species. In other embodiments, the at least one pluripotent stem cell and the diploid or tetraploid donor embryo are from differing species. In certain embodiments, the pluripotent stem cell and the diploid or tetraploid donor embryo are from differing species, but the differing species are related in some way. By way of example, the species are from the same genus, family, or order. By way of another example, the species are of a similar size (either as adults or as embryos).

In certain embodiments, the surrogate female is of the same species as the diploid or tetraploid donor embryo. In certain embodiments, the pluripotent stem cells, the surrogate mother, and the diploid or tetraploid donor embryo are all of the same species. In certain embodiments, the surrogate female is genetically related to the diploid or tetraploid donor embryo. In certain embodiments, the surrogate female is the same animal from whose ova the diploid or tetraploid donor embryo was generated. In certain embodiments, the surrogate female and the diploid or tetraploid donor embryo are from differing species, but the differing species are related in some way. By way of example, the species are from the same genus, family, or order. By way of another example, the species are of a similar size (either as adults or as embryos).

In certain embodiments, the surrogate female gestates the embryo for some period other than full term, and the embryo (the pluripotent stem cell-generated embryo) is harvested from the female for further study or analysis. In certain embodiments, the surrogate female animal gestates said embryo (the pluripotent stem cell-generated embryo) to term and delivers a pluripotent stem cell-generated animal.

In certain embodiments, the pluripotent stem cell is from an endangered species, and the diploid or tetraploid donor embryo is from a different species. In certain embodiments, the pluripotent stem cell is from an endangered species, and the diploid or tetraploid donor embryo is from a different but related species.

In certain embodiments, the pluripotent stem cell is genetically manipulated prior to combining said pluripotent stem cell with said donor embryo. In certain embodiments, genetically manipulated does not refer to the expression of reprogramming factors necessary to generate a pluripotent stem cell from a somatic cell. Rather, genetically manipulated refers to other manipulations of the genetic material of the cell, other than manipulations necessary to generate the pluripotent stem cell. In certain embodiments, genetically manipulating comprises correcting a genetic defect in the cell. In other embodiments, genetically manipulating comprises reducing immunocomplexity to reduce the chance of provoking an immune response in the host embryo or mother. In other embodiments, genetically manipulating comprises expression of a positive and/or negative selection marker and/or detectable label to allow identification and/or visualization of the pluripotent stem cell. In certain embodiments, the presence of a detectable label facilitate efficient identification of germ line cells that are genetically identical to the pluripotent cell. Once identified, such germ line cells can be combined using standard in vitro fertilization techniques to produce a clonal embryo.

(ii) Methods for Making iPS Cells:

Methods for making iPS cells by expressing or activating/inducing the expression of reprogramming factors are known in the art. Such methods can be used or readily modified to produce iPS cells from somatic cells obtained from virtually any species. Briefly, somatic cells are infected, transfected, or otherwise transduced with expression vectors expressing reprogramming factors. In the case of mouse, expression of four factors (Oct3/4, Sox2, c-myc, and Klf4) using integrative viral vectors was sufficient to reprogram a somatic cell. In the case of humans, expression of four factors (Oct3/4, Sox2, Nanog, and Lin28) using integrative viral vectors was sufficient to reprogram a somatic cell. However, expression of fewer factors or other reprogramming factors may also be sufficient. Additionally, the use of integrative vectors is only one mechanism for expressing reprogramming factors in the cells. Other methods including, for example, the use of non-integrative vectors can be used.

In certain embodiments, expression of one or more reprogramming factors is activated/induced by, for example, the use of small organic molecules, cytoplasm, or other agents.

Once the reprogramming factors are expressed or their expression is activated/induced in the cells, the cells are cultured. Over time, cells with ES characteristics appear in the culture dish. The cells can be picked and subcultured based on, for example, ES morphology, or based on expression of a selectable or detectable marker. The cells are cultured to produce a culture of cells that look like ES cells. These cells are putative iPS cells.

To confirm the pluripotency of the iPS cells, the cells can be tested in one or more assays of pluripotency. For examples, the cells can be tested for expression of ES cell markers; the cells can be evaluated for ability to produce teratomas when transplanted into SCID mice; the cells can be evaluated for ability to differentiate to produce cell types of all three germ layers.

In certain embodiments, the making of iPS cells is the goal. In such embodiments, the iPS cells can be used in vitro in screens to identify agents that promote proliferation, differentiation, or survival of the iPS cells. The iPS cells can be used to produce differentiated cell types, in vitro or in vivo. The iPS cells can be used to develop therapeutics.

In other embodiments, the making of iPS cells is in initial step in the production of pluripotent stem cell-generated embryos.

(iii) Methods for Making Diploid or Tetraploid Embryos

Diploid embryos can be generated by any well known methods. For example, diploid embryos can be generated in vitro by combining ova and sperm cells to produce a diploid embryo by in vitro fertilization. Alternatively, diploid embryos can be produced by sexual reproduction, and the resulting embryo can be harvested from the female before implantation in the uterus. For example, the embryo can be flushed from the fallopian tube.

Tetraploid embryos can be generated by any well known methods. Diploid embryos are the starting point for producing tetraploid embryos, and such diploids can be generated as described above. Tetraploidy can then be induced in vitro using well known mechanisms for inducing tetraploidy including chemical means, electrical stimulation, or mechanical means.

Pluripotent stem cells and diploid or tetraploid embryos can be combined using well known methods. For example, pluripotent stem cells can be injected or microsurgically implanted into the diploid or tetraploid embryo, or transferred into the embryo using electroporation. Alternatively, pluripotent stem cells and the diploid or tetraploid embryos can be aggregated and grown as cell aggregates. Regardless of the method used, the number of pluripotent stem cells combined with each embryo can be, for example, at least 1, 2, 3, 4, 5, 6, 7, 8, 9, or 10. In other embodiments, the number of pluripotent stem cells combined with each embryo can be, for example, greater than 10, greater than 20, greater than 50, or greater than 100.

(iv) Method for Identifying the Level of Cross-Species Flexibility of Reprogramming Factors

In another aspect, the invention provides a method for identifying the level of cross-species flexibility of reprogramming factors. In other words, the invention provides a method for assessing the level of species difference tolerated by reprogramming factors before they fail to function to reprogram a somatic cell to a pluripotent cell. The method comprises evaluating reprogramming factors in increasingly divergent or otherwise unrelated organisms, and evaluating at what point the reprogramming factor no longer induce reprogramming. If reprogramming factors from humans or mice fail to work in cells of a particular species, related reprogramming factors can be identified and cloned from other species that are more evolutionarily related to the organism in which reprogramming is desired. For example, cDNA libraries can be readily made from, for example, discarded testicular or other tissue of another species (e.g., bovine, ovine, pig, goat, horse, etc.). Reprogramming factors can be readily cloned using standard molecular biological approaches and used to induce reprogramming in cells of additional species, in the event that cDNA encoding human or mice factors fails to function across a certain evolutionary distance.

The invention contemplates all suitable combinations of any of the forgoing or following aspects and embodiments of the invention.

Exemplary methods and materials are described below, although methods and materials similar or equivalent to those described herein can also be used in the practice or testing of the present invention.

All publications, patents, patent publications and other references mentioned herein are incorporated by reference in their entirety.

Throughout this specification and claims, the word “comprise,” or variations such as “comprises” or “comprising” will be understood to imply the inclusion of a stated integer or group of integers but not the exclusion of any other integer or group of integers.

The following examples are intended to be illustrative and not limiting in any way.

### Exemplification

### EXAMPLE 1

### Generating iPS Cells Using Non-Mouse, Rodent Somatic Cells

Mice and human somatic cells have been reprogrammed to pluripotent stem cells (iPS cells) by expressing a set of reprogramming factors, culturing the cells, and selecting cells that have an embryonic stem cell appearance. The goal of this experiment is to make iPS cells from other non-mouse, rodent somatic cells. Rodent somatic cells are obtained from wild animals, laboratory animals, or from cell lines maintained by ATCC. The following are exemplary of the somatic cells that are used:
CCL-39 Hamster lung fibroblasts. This cell line was generated from adult, female cells. Prior to manipulation, the cells are propagated in McCoy's 5a medium (modified) with 1.5 mM L-glutamine adjusted to contain 2.2 g/L sodium bicarbonate. The medium contains 20% fetal bovine serum, which can optionally be reduced to 10% serum.
CCL-100 gerbil lung fibroblasts. This cell line was generated from 403 day old, female cells. Prior to manipulation, the cells are propagated in minimum essential medium (Eagle) with 2 mM L-glutamine and Earle's BSS adjusted to contain 1.5 g/L sodium bicarbonate, 0.1 mM non-essential amino acids, and 1.0 mM sodium pyruvate. The medium contains 10% fetal bovine serum.
CRL 1926 plantain squirrel embryonic fibroblasts. This cell line was generated from embryonic material. Prior to manipulation, the cells are propagated in 45% Dulbecco's modified Eagle's medium, 45% Ham's F12 medium, and 10% fetal bovine serum. The medium is supplemented with 10 ng/ml EGF, 0.005 mg/ml insulin, 5 ng/ml selenium, and 0.005 mg/ml transferrin.
CCL-158 Guinea Pig fibroblasts. This cell line was generated from adult female tissue. Prior to manipulation, the cells are propagated in Ham's F12K medium with 10% fetal bovine serum.

Non-murine rodent somatic cells are obtained. iPS cells are generated by expressing in the somatic cells murine (closely related genetically) reprogramming factors (Oct3/4, c-myc, Sox2, and KLF4). The reprogramming factors are expressed as previously described using viral vectors to infect the somatic cells.

Once obtained, putative iPS cells are tested to confirm pluripotency. Note, putative cells are selected based on morphology and/or based on expression of a selectable or detectable marker (LacZ, GFP, etc.). Putative iPS cells are analyzed for expression of ES cell specific markers such as Oct-4, Nanog, Rex-1, SSEA antigens, alkaline phosphatase. Putative iPS cells are also analyzed to confirm that germ line transmission in chimeric embryos is possible. Putative iPS cells are also analyzed to confirm that they can differentiate to produce cell types of all three germ layers and/or that they form teratomas when transplanted into SCID mice.

### EXAMPLE 2

### Reprogramming of Somatic Cells from an Endangered Species to Induced Pluripotent Stem Cells (iPS)

Somatic cells from an endangered species are used to produce iPS cells using methods known in the art to produce iPS cells from, for example, mice and human somatic cells. The somatic cells are infected, transfected, or otherwise transduced with reprogramming factors (1, 2, 3, 4, 5, or 6 factors), cultured, and cells with an embryonic stem cell appearance (prominent nucleoli, small amount of cytoplasm, etc) are selected and subcultured to produce a population of iPS cells. Once produced, the iPS cells are tested to confirm that they are pluripotent. For example, (i) the cells are transplanted to a SCID mouse to assess teratoma formation; (ii) the cells are differentiated to confirm that they can differentiate along all three germ layers; (iii) the cells are analyzed for expression of embryonic stem cell markers (e.g., Oct4, alkaline phosphatase, REX1, etc).

Following these procedures, iPS cells are produced from somatic cells from an endangered species. If constructs capable of expressing reprogramming factor genes from the endangered species are already available, such constructs are used. In this way, there is no divergence between the genes encoding the reprogramming factors and the somatic cells.

If such constructs do not yet exist, human or mouse constructs are used. It is possible, however, that the level of divergence between the genes of the endangered species and human or mouse is too large, and that this divergence will prevent successful reprogramming. If reprogramming is not successful using human or mouse constructs, genes encoding the orthologues of these reprogramming factors will be isolated from a cDNA library made from tissue from the endangered species of interest. These species-specific reprogramming factors are then used to produce iPS cells. If a cDNA library cannot be readily produced from tissues from the endangered species, than a species that is as close to the endangered species, in an evolutionary or taxonomic sense, is chosen and used to make a cDNA library from which genes encoding the reprogramming factors are isolated. When using a related species, the species is ideally one that (i) is easily available for captivity so that suitable tissue samples can be obtained; (ii) is close to the species of interest in an evolutionary or taxonomic sense—for example, of the same genus, family, or order.

A cDNA library is made from a tissue that expresses reprogramming factors. Testis is one example of such a tissue. Thus, cDNA libraries are generated from testis or from other readily available tissue that expresses reprogramming factors.

Reprogramming factors are isolated from the cDNA library by direct amplification using primers designed based on the known sequences of various reprogramming factors. Once reprogramming factors from the cDNA library are amplified, the sequences are subcloned into suitable vectors for delivery to somatic cells.

### EXAMPLE 3

### Generating a Giant Panda Embryo

Giant panda are an endangered species. Maintaining this species is difficult because the giant panda does not breed well, particularly in captivity. Thus, if the numbers of giant panda continue to diminish, natural breeding programs will not be sufficient to help protect the species from extinction.

Although giant panda are large animals, their embryos are quite small. In fact, the giant panda embryos and neonates are comparable in size to the embryos and neonates of rats.

A fibroblast sample from a giant panda is obtained. The sample is used to generate pluripotent stem cells by overexpressing mouse, human, rat, or panda reprogramming factors (1, 2, 3, 4, 5, or 6 factors) selected from Oct4, Sox2, Nanog, Lin28, c-Myc, and Klf4. If necessary, the cDNAs encoding the panda ortholog of the reprogramming factors are isolated from a panda cDNA library.

Once generated, the pluripotency of the panda pluripotent stem cells is evaluated to confirm that the cells are pluripotent stem cells. The pluripotent stem cells are combined with a rat tetraploid embryo. The rat embryo is generated in vitro and tetraploidy is induced in vitro. The tetraploid rat embryo comprising the panda pluripotent stem cells is cultured in vitro and then the developing embryo is transferred to a pseudo pregnant rat female. The pseudo pregnant rat female gestates the developing embryo to or near term, and the embryo is delivered. Because the donor rat embryo is tetraploid, the resulting pluripotent stem cell-generated embryo is genetically related to the panda pluripotent stem cells. In other words, the pluripotent stem cell-generated embryo is a giant panda embryo.

Other donors can similarly be used including, but not limited to, a rabbit or a hamster.

The following are hereby incorporated by reference in their entirety:

Takahashi et al. (2007) Nat Protoc. 2(12): 3081-9.

Maherali et al. (2007) Cell Stem Cell 1: 55-70.

Hanna et al. (2007) Science, 6 Dec. 2007, 10.1126/science.1152092 (see also www.sciencexpress.org).

Meissner et al. (2007) Nature Biotechnology 25: 1177-1181.

Mikkelsen et al. (2007) Nature, Vol 448, 2 Aug. 2007, doi:10.1038/nature06008.

Wernig et al. (2007) Nature, Vol 448, 19 Jul. 2007, doi:10.1038/nature05944.

Yu et al. (2007) Science, 20 Nov. 2007, 10.1126/science.1151526 (see also www.sciencexpress.org).

Vogel and Holden (2007) Science 318: 1224-1225.

Takahashi et al. (2007) Cell 131: 861-872.

Wang et al. (1997) Mechanisms of Development 62: 137-145.

Wang et al. (2004) Developmental Biology 275: 192-201.

Li et al. (2005) Reproduction Research 130: 53-59.

It is to be understood that the foregoing description is merely a disclosure of particular embodiments and is in no way intended to limit the scope of the disclosure. All operative combinations of any of the foregoing aspects and embodiments are contemplated and are within the scope of the invention. Other possible modifications will be apparent to those skilled in the art and all modifications will be apparent to those in the art and all modifications are to be defined by the following claims. The invention contemplates all suitable combinations of any of the forgoing or following aspects and embodiments of the invention.

1. A method of producing a pluripotent stem cell-generated embryo, comprising
providing at least one pluripotent stem cell;
combining the at least one pluripotent stem cell with a diploid or tetraploid donor embryo to generate a diploid or tetraploid donor embryo comprising said at least one pluripotent stem cell;
culturing said diploid or tetraploid donor embryo comprising said at least one pluripotent stem cell; and
transferring said diploid or tetraploid donor embryo comprising said at least one pluripotent stem cell to a surrogate female animal, whereby said surrogate female animal gestates said embryo to produce a pluripotent stem cell-generated embryo.

2. The method of claim 1, wherein the at least one pluripotent stem cell is an induced pluripotent cell, embryonic stem cell, or embryo-derived cell.

3. (canceled)

4. The method of claim 1, wherein the donor embryo is a diploid donor embryo and the pluripotent stem cell-generated embryo is a chimeric embryo comprising cells genetically identical to the pluripotent stem cell and cells genetically identical to the donor embryo.

5. The method of claim 4, further comprising
identifying one or more gametes from the pluripotent stem cell-generated embryo, which gametes are genetically identical to the pluripotent stem cell, and
using said gametes for in vitro fertilization to produce an embryo genetically identical to the pluripotent stem cell.

6. The method of claim 5, further comprising
transferring said embryo that is genetically identical to said pluripotent stem cell to a surrogate female to gestate said embryo.

7. The method of claim 5, wherein the one or more gametes are obtained from the same pluripotent stem cell-generated embryo.

8. The method of claim 5, wherein the one or more gametes are obtained from different pluripotent stem cell-generated embryos, but which pluripotent stem-cell generated embryos were produced using genetically identical pluripotent stem cells.

9. The method of claim 5, wherein the pluripotent stem cell is labeled to facilitate identification of the one or more gametes.

10. The method of claim 1, wherein the donor embryo is a tetraploid donor embryo and substantially all of the cells of the pluripotent stem cell-generated embryo are genetically identical to the pluripotent stem cell.

11. The method of claim 1, wherein the donor embryo is a tetraploid donor embryo and the pluripotent stem cell-generated embryo is genotypically identical to the pluripotent stem cell.

12. The method of claim 1, wherein the at least one pluripotent stem cell is an induced pluripotent stem cell produced using at least one donor somatic cell.

13. The method of claim 12, wherein the at least one donor somatic cell is selected from embryonic, fetal, neonatal, juvenile, or adult cells.

14. (canceled)

15. The method of claim 13, wherein the at least one donor somatic cell is a fibroblast.

16. The method of claim 1, wherein the at least one pluripotent stem cell is a human cell.

17. The method of claim 1, wherein the at least one pluripotent stem cell and the diploid or tetraploid donor embryo are from the same species.

18. The method of claim 1, wherein the at least one pluripotent stem cell and the diploid or tetraploid donor embryo are from differing species.

19. The method of claim 1, wherein the surrogate mother is of the same species as the diploid or tetraploid donor embryo.

20. The method of claim 1, wherein the surrogate female animal gestates said embryo to term and delivers a pluripotent stem cell-generated animal.

21. The method of claim 1, wherein the at least one pluripotent stem cell is from an endangered species, and the diploid or tetraploid donor embryo is from a different species.

22. (canceled)

23. (Cancelled)

24. The method of claim 1, wherein the at least one pluripotent stem cell, diploid or tetraploid embryo, and/or surrogate mother is selected from the group consisting of: goats, sheep, horses, pigs, cattle, bison, elk, alpaca, llama, emu, cattle, tigers, lions, bears, cheetahs, jaguars, elephants, giraffes, and zebras.

25-30. (canceled)
